(19) United States
(12) Patent Application Publication
(10) Pub. No.: US 2017/0119658 A1
(43) Pub. Date: May 4, 2017

(54) PREPARATION FOR ENHANCED FINGERPRINT IMAGE FORMATION ON A TRANSPARENT SURFACE OF A LIVE SCAN DEVICE

(71) Applicant: Justin Turvey, Pembroke, MA (US)

(72) Inventor: Justin Turvey, Pembroke, MA (US)

(21) Appl. No.: 14/929,365

(22) Filed: Nov. 1, 2015

(51) Int. Cl. A61K 8/92; A61K 8/31; A61Q 19/00; G06K 9/00

(52) U.S. Cl. CPC A61K 8/927; G06K 9/00013; G06K 9/00073; A61K 8/31; A61K 8/922; A61Q 19/007

(57) ABSTRACT

A preparation is provided for enhancement of imaging of fingerprints and palm prints acquired using a Live Scan device. The preparation is solid at room temperature, even in hot climates, and can be consistently applied. The preparation is a mixture of: 20%-70% beeswax; 1.0%-50% squalane; 1.0%-10% jojoba oil; and 0.1%-5.0% tea tree oil. The mixture can be advantageously molded into a shape that facilitates application, such as the shape of a deodorant stick or lip balm. Preferably, the preparation consists of: 60% beeswax; 30% squalane; 9% jojoba oil; and 1% tea tree oil. The preparation moisturizes skin to facilitate higher quality images on AFIS or IAFIS Live Scanners, and includes an antimicrobial to maximize sanitary conditions, includes no alcohol to be safe to use around a breathalyzer, is non-toxic for use in jails and mental institutions, and will not stain clothing.

### FIELD

This invention relates generally to finger printing technology, and more particularly to finger print image acquisition.

### BACKGROUND

The main purpose of law enforcement at a crime scene is to document, preserve, and collect evidence. An important part of such evidence is the collection of latent fingerprints. These prints are used to identify individuals who were at the crime scene. For this process to work, there must be a database or library of known fingerprints on file. To compile such a database, the fingerprints (and now palm prints) of every person arrested for a criminal offence are taken during the booking process. The equipment used to capture the prints is predominantly electronic with the use of a “Live Scan” device, having a transparent glass surface for acquiring high-resolution images of fingers and palms pressed against the transparent glass surface.

Live Scan fingerprinting enables capture of fingerprints and palm prints electronically, without the need for the more traditional method of ink and paper.

In the US, most law enforcement agencies use Live Scan technology as their primary tool in the recognition of persons. Live Scan is commonly used for criminal booking, sexual offender registration, civil applicant, and background checks. In the UK, many major police custody suits are now equipped with Live Scan machines, which enable suspects' fingerprints to be instantly compared with a national database.

The old method using ink and paper for obtaining fingerprints used ink to transfer the elevated ridge detail of a person's hand to paper that was later scanned into the Automated Fingerprint Identification System (AFIS) database. The new Live Scan method scans the hand directly into the system without ink. The same Live Scan machines are also used to acquire and record fingerprints of people for jobs or licenses, such as firearms permits, and the problems described below apply to these uses as well.

A Live Scan device captures images of only the part of the hand (such as a finger or a palm) that makes contact with the glass, and produces an image of just the raised ridges, the raised ridges being used to identify the person. However, if there is interference with the skin making full contact with the glass of the live scanner device, a poor image of the identifying ridges will result.

With the present use of Live Scan devices to capture the image, the key issues have become: skin quality of the person being fingerprinted, and substances that may be found on the skin of the person being fingerprinted. There are a few problems that can arise when attempting to acquire an image of a fingerprint or a palm print using a live scanner device:

1. Images can be Too Light

This can happen due to lack of moisture on the skin, which causes the image to show up very light, and in some cases the image can be so light as to be unreadable.

2. Images can be Too Dark

Excess sweat, water, oil, or any moisture can cause the image to darken to the point where the ridge lines come together and produce a black spot in the image, or blacking out an entire area of the image.

3. Missing Areas in the Image—

When skin is peeling, or has a callused area such areas can show up blank, or too light to see. Finger joint areas that are slightly recessed but nevertheless have ridge lines that can provide information are often not imaged at all.

4. Inconsistent Image Darkness—

If the entire area scanned does not have a consistent moisture level, then the image will have light and dark areas that make the image of the print hard to see and/or hard to understand.

If any one of the issues above are present with enough severity, then the scan will be rejected and the scan must be redone. When dealing with an uncooperative subject, or someone who is under the influence of drugs or alcohol, this becomes not only an inconvenience, but can become an officer safety issue if the subject becomes agitated that the process is taking too long.

Attempted Solutions

1. Images can be Too Light—

Plain or distilled water can be applied with a fine mist from a spray bottle. When water is used on some live scan devics, it is too difficult to apply without over-applying and causing the entire area to turn black, and so a lot of time is spent waiting for the skin to dry enough to proceed. Lotions or oils by themselves are nearly impossible to apply without using too much, thereby causing the same black out effect. Other products, such as Ridge Builder™ or EZ Scan™ are in liquid form, and are therefore easily spilled and require thorough washing and then drying of the hands prior to and after application. Some of the current products available have a large amount of alcohol (as much as 98.2%), which can be dangerous to keep in a jail or a prison where the product could be consumed easily or splashed into an Officer's eyes due to it being in liquid form. High alcohol content can also pose a serious problem when the breathalyzer is being used during a booking procedure.

2. Images can be Too Dark—

This issue will remain constant even without the use of an additional product. This issue can be solved by cleaning the hands prior to scanning, thereby drying out the skin, possibly resulting in images that can be too light.

3. Missing Areas in the Image—

To have the Live Scan device pick up on the missing areas from a callused area of the hand, the same techniques for dealing with light images have been used, and similar problems are experienced. Moreover, joint areas still fail to be imaged.

4. Inconsistent Image Darkness—

The problem posed by products that are currently available is that it is difficult to apply such products evenly so as to produce consistent image darkness. When a liquid product is applied, the goal is to apply the product evenly. It if is not applied evenly, the image will be dark in some spots while light in others. When water is used, three outcomes are possible. One is that if the person has any lotion, contaminants, dirt, or oil on their hand, the water mixes with them to produce a mud-like substance, resulting in a mess on the glass, thereby producing unusable images. Also, the water is absorbed into some areas of the skin faster than others, and in some areas the water remains on the surface and causes blotchy images. Further, the water must be applied several times on each hand because it evaporates quickly, which wastes time and becomes frustrating to the person being printed.

The problems introduced by the use of water, or products presently available, is that while in some cases they may work, they work only inconsistently throughout a fingerprinting session, and depend on the person to be fingerprinted being willing to cooperate throughout the session, which cannot be relied upon.

Latent fingerprints from crime scenes that are collected by law enforcement officers worldwide are not always perfect, and are not always complete prints. Sometimes, all the print examiner has to work with is a small portion of a suspect's fingerprint. Consequently, if the library of fingerprints on file is of poor quality, or has missing areas due simply to a bad scan, then there is a greater chance that no match will be found, and a crime could go unsolved.

### SUMMARY

The preparation of the invention enables Automated Fingerprint Identification Systems (AFIS) and Integrated Automated Fingerprint Identification Systems (IAFIS) Live Scan machines to capture more detailed and complete fingerprint and palm print images, usually on the first attempt. The preparation of the invention provides clear, crisp, dark, and detailed images of fingers and palms pressed against the glass of a Live Scan machine. The preparation of the invention can reveal more accurate minutiae and discernable patterns, and this additional data is easily incorporated into the AFIS database. The preparation of the invention is a waxy solid that is most advantageously dispensed in stick form, that can be easily applied evenly to fingers and palm, and that can remain on the skin for an entire session of fingerprinting and palm printing using a Live Scan machine. It is good for the skin, and does not need to be washed off or removed after use. Use of the preparation reduces the amount of rejected prints, and increases the number of high quality prints.

Databases of fingerprints are only as good as the images scanned into the system. If a scanned image of a fingerprint bears too many defects and gaps (missing minutia), the scanned image is useless in that it cannot be used to identify an individual.

When dealing with applications or licensing issues, the ability to capture a better image of a person's fingerprint can lessen the chance of a rejected application, and saves time and money. More importantly, this is not a problem where the solution can just save a few dollars to the user, or save time on doing a task—improved print image quality due to the use of the preparation of the invention could save lives. With higher quality fingerprint images in the database, a person who commits a crime will have a better chance of being apprehended before they can offend again.

Finger joint areas that are slightly recessed but nevertheless have ridge lines can now be imaged, thereby providing further information for use in identification. In particular, the area below the first crease of the fingertip is almost never present on a scan without some sort of product applied. While other products can be used to enhance images of this portion of the finger, the same problems arise, such as alcohol content, toxic materials, evaporation, uneven application, over application, etc. The preparation of the invention avoids all of these problems. Good imaging of this portion of the finger is very important when matching latent fingerprints from crime scenes that may only include information from this portion of the finger, so without the use of an image enhancement formulation, there would be no data to compare with the original scan.

Use of the preparation of the invention on dry skin results in darker print images that would typically be too light.

Use of the preparation of the invention prior to scanning provides an even contrast over an entire scan image.

Use of the preparation of the invention prior to scanning allows users to attain passing scans more often with fewer rejected scans.

Use of the preparation of the invention prior to scanning enables users to obtain highly optimized images of fingerprints and palm prints.

Use of the preparation of the invention prior to scanning results in scan images having crisp images of print ridge detail.

Use of the preparation of the invention avoids over-application by being presented to the fingers and palm in solid form, unlike all other known preparations for enhancement of Live Scan images.

A general aspect of the invention is a preparation for enhancement of imaging of finger prints and palm prints on a transparent surface of a live scan device. The preparation includes a mixture of: beeswax; squalane; jojoba oil; and tea tree oil.

In some embodiments, the preparation consists of a mixture of: 20%-70% beeswax; 1.0%-50% squalane; 1.0%-10% jojoba oil; and 0.1%-5.0% tea tree oil.

In some embodiments, the preparation consists of a mixture of: 60% beeswax; 30% squalane; 9% jojoba oil; and 1% tea tree oil.

In some embodiments, the mixture is solid at room temperature, and is molded into a shape that facilitates application to at least one of a finger and a palm of a person.

In some embodiments, the beeswax is replaced by at least one of: Carnuba Wax, Candelilla Wax, Pola Wax, Rice Bran Wax, Soy Wax, emulsifying wax NF (e-wax).

In some embodiments, the squalane is replaced by one of: glycerin, sorbitol, sodium hyaluronate, urea, alpha hydroxy acids.

In some embodiments, the squalane is replaced by a mixture of: Hydrogenated Poly (C6-14 Olefin); Olea Europa (Olive) Fruit Extract; Beta-Sitosterol; and Mixed Tocopherols.

In some embodiments, the jojoba oil is replaced by at least one of: olive oil, canola oil, grapeseed oil, safflower oil, argan oil, coconut oil, babassu oil, camellio oil, sunflower oil, flaxseed oil, vegetable oil, avocado oil.

In some embodiments, the jojoba oil is replaced by a mixture of: Limnanthes Alba (Meadowfoam) Seed Oil; Hydrogenated Poly (C6-14 Olefin); Olea Europa (Olive) Fruit Extract; Beta-Sitosterol; and Mixed Tocopherols.

In some embodiments, the tea tree oil is replaced by one of: thymol crystals, Zinc, Urea.

Another general aspect of the invention is a preparation for enhancement of imaging of finger prints and palm prints on a transparent surface of a live scan device, where the preparation consists of a mixture of: 60% beeswax; 30% squalane; 9% jojoba oil; and 1% tea tree oil.

In some embodiments, the mixture is solid at room temperature, and is molded into a shape that facilitates application to at least one of a finger and a palm of a person.

In some embodiments, the beeswax is replaced by one of: Candelilla Wax, Rice Bran Wax, Soy Wax.

In some embodiments, the squalane is replaced by a mixture of: Hydrogenated Poly (C6-14 Olefin); Olea Europa (Olive) Fruit Extract; Beta-Sitosterol; and Mixed Tocopherols.

In some embodiments, the jojoba oil is replaced by a mixture of: Limnanthes Alba (Meadowfoam) Seed Oil; Hydrogenated Poly (C6-14 Olefin); Olea Europa (Olive) Fruit Extract; Beta-Sitosterol; and Mixed Tocopherols.

In some embodiments, the tea tree oil is replaced by one of: thymol crystals, Zinc, Urea.

Another general aspect of the invention is a preparation for enhancement of imaging of finger prints and palm prints on a transparent surface of a live scan device, the preparation being a solid mixture of: a moisturizer to swell the ridges of the skin; an oil augmenter to add to the oil on the ridges of the skin, which are to be pressed against the glass of the Live Scan machine to create dark image lines; and a waxy matrix to be admixed with the moisturizer and the oil augmenter so as to keep the entire mixture solid at room temperatures, while facilitating transfer at least the moisturizer and the oil augmenter to the skin of fingers and palms to be printed when the solid mixture is applied thereto.

In some embodiments, the preparation further includes an antimicrobial agent to prevent the moisturizer and the oil augmenter from spreading and promoting the growth of microbes.

In some embodiments, the oil augmenter is jojoba oil.

In some embodiments, the moisturizer is squalane.

### DETAILED DESCRIPTION

The preparation of the invention has been formulated to have the following attributes:

Moisturizes the skin so as to allow for higher print image quality on an AFIS Live Scan machine.

Includes antimicrobial ingredients to maximize sanitary conditions.

Has a high melting point so it will remain solid so that it can work effectively even in hot climates.

Includes no alcohol so that it is safe to use around a breathalyzer without distorting test results.

Contains no harmful or toxic ingredients, and so it is safe for use in secure locations, such as jails, prisons, booking areas, or mental institutions.

Will not stain clothing.

Has all plastic packaging that is safe for use in jails or police stations.

Can be packaged and used in “stick” form, like a deodorant stick or a lip balm.

Will not spill, splash, leak, or dry out.

Can dispense more uses per stick than can be dispensed by containers of competing preparations.

The above attributes are achieved by an elegant formulation that serves to:

1. Swell ridges on the surface of the fingers and palm using a moisturizing agent. Even worn ridges swell enough so as to allow better detail to be captured by a Live Scan machine.

2. Replace or augment the natural oils typically found on the skin of fingers and palms, resulting in images of sufficient darkness even in dry or callused areas, without leaving excess residue. This is accomplished by using an oil found in nature that is very similar to sebum, the oily substance normally found on skin.

3. Inhibit the growth and spread of germs by including an antimicrobial agent, without the use of alcohol that can distort the results of a breathalyzer test.

4. Dispense as a waxy solid in the form of a convenient easily controllable applicator stick, resembling a solid deodorant stick or solid lip balm dispenser.

To meet these objectives, a preferred formulation includes a mixture of:

Squalane to moisturize and thereby swell the ridges of the skin;

Jojoba Oil to augment the oil on the ridges of the skin, which are to be pressed against the glass of the Live Scan machine to create dark image lines;

Tea Tree Oil to serve as an antimicrobial agent to prevent the moisturizer and oil augmenter from supporting or promoting the growth of microbes; and

Beeswax to be admixed with the moisturizer, the oil augmenter, and the antimicrobial so as to keep the entire mixture solid at room temperatures, while transferring at least the moisturizer and the oil augmenter to the skin of fingers and palms to be printed when the solid mixture is applied thereto.

Ideally, the formulation will be a mixture consisting of:

| | |
|---|---|
| Beeswax | 60% |
| Squalane | 30% |
| Jojoba Golden Oil | 9% (The “Golden Oil” is the Jojoba oil that is first |
| | produced from the extraction from seed |
| | before it is refined. 100% cold pressed |
| | jojoba -- virgin jojoba oil -- is a clear golden color.) |
| Tea Tree Oil | 1% |

This mixture is a solid with a melting point of about 122 degrees F. Thus, the finished product will not melt at room temperature (25 degrees C.), and that is why it can be formed as a stick. Beeswax has a melting temperature of around 62 degrees C. The other three ingredients are liquid oils at room temperature (25*C).

The effective range (Min-Max) of each ingredient percentage that would still result in an effective formulation is:

| | |
|---|---|
| Beeswax | 20%-70% |
| Squalane | 1.0%-50% |
| Jojoba Golden Oil | 1.0%-10% |
| Tea Tree Oil | 0.1%-5.0% |

Beeswax has a high binding strength and exhibits excellent ability to emulsify, improve structure, provide oil retention, and facilitate mold release for stick applications. Suitable substitutes for Beeswax include: Carnuba Wax, Candelilla Wax, Pola Wax, Rice Bran Wax, Soy Wax, emulsifying wax NF (e-wax), or any other wax or mixture of waxes that has a similar melting point that can hold the other ingredients in suspension, maintain solid form during storage and use, and allow a controlled and even application of the mixture to be applied to the skin. Beeswax was chosen also because it is non-toxic and inexpensive, and can hold the other ingredients in a suspension while allowing a precise and small measured amount of the other ingredients to be applied to the skin, consistently with each application.

Processed beeswax was chosen due to it being inexpensive, easy to procure, as well as providing consistent samples that do not vary due to other outside factors. For example, organic beeswax can have inconsistencies due to being unrefined.

If the beeswax or substitute was not included in the formulation of the invention, the other ingredients of the formulation would be in liquid form, and consequently it would be difficult to control the application of the formulation to the skin precisely, and it would be difficult to limit the amount of the formulation applied to the skin, and it would be difficult to prevent leaks and spills, and thus the formulation without beeswax or an equivalent would not work as well as the solid form.

Squalane is an excellent moisturizer, helping to swell the ridges of the skin with moisture, soften the skin, and restore natural protective barrier properties against environmental stresses. It stimulates healing and soothes the skin. One substitute for Squalane is a mixture of: Hydrogenated Poly (C6-14 Olefin); AND Olea Europa (Olive) Fruit Extract; AND Beta-Sitosterol; AND Mixed Tocopherols. Other substitute moisturizing agents that are able to remain in suspension with the other ingredients, and provide moisture in a measured and even amount to the skin can also be used. Squalane was chosen because it is an excellent moisturizer and emollient. It is important to provide moisture to the fingers and palm to be printed so as to plump and swell key features of a person's skin surface in a controlled manner. This results in more contact of the ridges with the glass of the Live Scan machine, and less contact of the non-raised portion of the skin, resulting in more detail acquired by the Live Scan device. This particularly enhances the image of skin that is worn or elderly, and therefore relatively smooth prior to application of an embodiment of the formulation of the invention.

Suitable substitute for squalane can be any other moisturizing agent, especially any ingredient that contains a humectant, such as glycerin, sorbitol, sodium hyaluronate, urea, and/or alpha hydroxy acids. Some squalane is derived from fish. A favorable form of squalane is derived from the olive plant, or other suitable plant.

However squalane was chosen because it is found naturally in the skin, the ease and speed of absorption into the skin, as well as the absence of alcohol, which other known formulations actually include, even though the presence of alcohol in known formulations can interfere with breathalyzer testing.

If squalane (or other suitable moisturizer) was not included in the formulation of the invention, the amount of oil used (whatever that oil may be), would have to be increased to the point where it would be too soft and too greasy to use. Alternatively, if the oil percentage was not increased, the lack of squalane would leave the beeswax (or it's substitute) way too hard and difficult to apply. Consequently, such a formulation without squalane or equivalent moisturizer would not work well enough to swell the ridges of the skin on the finger, and so would fail to enhance imaging of the fingerprint and/or palm print.

Jojoba Oil has very good effects on the skin, acting as a moisturizer and emollient agent to improve skin elasticity and suppleness. Also, Jojoba oil is chemically very similar to sebum as naturally found in skin. Thus, it augments the oiliness of skin in a very natural way. Jojoba contains natural tocopherols to minimize oxidation, and thereby reduces rancidity caused by lipid peroxidation. Jojoba oil can be replaced by a mixture of: Limnanthes Alba (Meadowfoam) Seed Oil; Hydrogenated Poly (C6-14 Olefin); Olea Europa (Olive) Fruit Extract; Beta-Sitosterol; and Mixed Tocopherols, or any other oil or oily mixture that is able to remain in suspension with the other ingredients and provide oil augmentation in a measured and even amount to the skin. Jojoba oil was chosen because of close chemical similarity with the oils found in sebum that is secreted when a person sweats. Thus, Jojoba oil adds to or replaces oils normally secreted by the body, and thereby darkens images acquired by a Live Scan machine.

Suitable substitutes for jojoba oil can be any other oil able to be held in suspension with the other ingredients that can be applied evenly in controlled amounts to the skin, thereby allowing a darker image to result. Suitable substitute oils include one or a mixture of: olive oil, canola oil, grapeseed oil, safflower oil, argan oil, coconut oil, babassu oil, camellio oil, sunflower oil, flaxseed oil, vegetable oil, avocado oil.

However, jojoba oil was chosen as the preferred oil because it is relatively lightweight and thin, as well as almost exactly replicating the properties of oils found in sebum, a substance commonly found in and on the skin.

If Jojoba oil or an equivalent was not included in the formulation, the formulation would be effective, but not as effective as the complete formulation, since the oil in the skin as augmented by the jojoba oil is what darkens the image, thereby providing more print information. However, there may be enough oil in the other ingredients of the formulation of the invention, such as Tea tree oil or squalane, or equivalents, that the formulation without jojoba oil or an equivalent could still provide modest performance.

Tea Tree Oil is a proven anti-bacterial, anti-fungal, anti-viral, and anti-inflammatory. Applications include acne, wounds, Methicillin-resistant Staphylococcus aureus (MRSA), dandruff, and hand/body washes. Tea tree oil was chosen because it is non-toxic, and promotes a sanitary condition on the stick of the mixture without harsh chemicals or alcohol. Although thymol crystals could also be used, thymol crystals are more expensive and not readily available.

Tea tree oil substitutes can be any antimicrobial agent able to be added and held in suspension that can discourage the growth of bacteria and other germs. Suitable substitutes include: thymol crystals, Zinc, and/or Urea.

However, Tea tree oil was chosen due to its easy availability and lower cost.

If an antimicrobial agent was not included in the formulation, the formulation would still work, but there would be more of a risk of the spread of disease due to bacteria or other disease organisms due to contact of the stick with the skin of many persons.

To use the preparation of the invention in solid stick form, first remove the cap from the dispenser (such as found in many solid deodorants, or solid lip balms) to expose an end of the solid stick of the preparation. Apply the end of the solid stick to the surface to be printed of each finger, and optionally to the palm to be printed. For example, one can run the stick up one side of a finger, and down the other side, repeating for each finger. The preparation can be applied to the palm using a zig-zag pattern, for example. Next, using a glove that is resistant to degradation by oil, rub all surfaces with a gloved thumb so as to ensure that the preparation is evenly spread over all surfaces to be printed using the Live Scan machine.

Although the best form to present the preparation of the invention to the fingers is a solid stick of the formulation, the formulation can also be contained in a shallow container with an opening large enough to enable application to each finger by wiping the pad of each finger on the surface of the preparation in the shallow container. The container is shallow so that the walls of the container do not interfere with the wiping action of a finger over the solid surface of the preparation. Alternatively, the container can be sized so that it can accommodate an entire hand to be printed, including all fingers and the palm.

Other modifications and implementations will occur to those skilled in the art without departing from the spirit and the scope of the invention as claimed. Accordingly, the above description is not intended to limit the invention, except as indicated in the following claims.

What is claimed is:

1. A preparation for enhancement of imaging of finger prints and palm prints on a transparent surface of a live scan device, the preparation including a mixture of:
beeswax;
squalane;
jojoba oil; and
tea tree oil.

2. The preparation of claim 1, the preparation consisting of a mixture of:
20%-70% beeswax;
1.0%-50% squalane;
1.0%-10% jojoba oil; and
0.1%-5.0% tea tree oil.

3. The preparation of claim 1, the preparation consisting of a mixture of:
60% beeswax;
30% squalane;
9% jojoba oil; and
1% tea tree oil.

4. The preparation of claim 1, wherein the mixture is solid at room temperature, and is molded into a shape that facilitates application to at least one of a finger and a palm of a person.

5. The preparation of claim 1, wherein beeswax is replaced by at least one of: Carnuba Wax, Candelilla Wax, Pola Wax, Rice Bran Wax, Soy Wax, emulsifying wax NF (e-wax).

6. The preparation of claim 1, wherein squalane is replaced by one of:
glycerin, sorbitol, sodium hyaluronate, urea, alpha hydroxy acids.

7. The preparation of claim 1, wherein squalane is replaced by a mixture of: Hydrogenated Poly (C6-14 Olefin); Olea Europa (Olive) Fruit Extract; Beta-Sitosterol; and Mixed Tocopherols.

8. The preparation of claim 1, wherein jojoba oil is replaced by at least one of: olive oil, canola oil, grapeseed oil, safflower oil, argan oil, coconut oil, babassu oil, camellio oil, sunflower oil, flaxseed oil, vegetable oil, avocado oil.

9. The preparation of claim 1, wherein jojoba oil is replaced by a mixture of: Limnanthes Alba (Meadowfoam) Seed Oil; Hydrogenated Poly (C6-14 Olefin); Olea Europa (Olive) Fruit Extract; Beta-Sitosterol; and Mixed Tocopherols.

10. The preparation of claim 1, wherein tea tree oil is replaced by one of: thymol crystals, Zinc, Urea.

11. A preparation for enhancement of imaging of finger prints and palm prints on a transparent surface of a live scan device, the preparation consisting of a mixture of:
60% beeswax;
30% squalane;
9% jojoba oil; and
1% tea tree oil.

12. The preparation of claim 11, wherein the mixture is solid at room temperature, and is molded into a shape that facilitates application to at least one of a finger and a palm of a person.

13. The preparation of claim 11, wherein beeswax is replaced by one of: Candelilla Wax, Rice Bran Wax, Soy Wax.

14. The preparation of claim 11, wherein squalane is replaced by a mixture of: Hydrogenated Poly (C6-14 Olefin); Olea Europa (Olive) Fruit Extract; Beta-Sitosterol; and Mixed Tocopherols.

15. The preparation of claim 11, wherein jojoba oil is replaced by a mixture of: Limnanthes Alba (Meadowfoam) Seed Oil; Hydrogenated Poly (C6-14 Olefin); Olea Europa (Olive) Fruit Extract; Beta-Sitosterol; and Mixed Tocopherols.

16. The preparation of claim 11, wherein tea tree oil is replaced by one of: thymol crystals, Zinc, Urea.

17. A preparation for enhancement of imaging of finger prints and palm prints on a transparent surface of a live scan device, the preparation being a solid mixture of:
a moisturizer to swell the ridges of the skin;
an oil augmenter to add to the oil on the ridges of the skin, which are to be pressed against the glass of the Live Scan machine to create dark image lines; and
a waxy matrix to be admixed with the moisturizer and the oil augmenter so as to keep the entire mixture solid at room temperatures, while facilitating transfer at least the moisturizer and the oil augmenter to the skin of fingers and palms to be printed when the solid mixture is applied thereto.

18. The preparation of claim 17, further including an antimicrobial agent to prevent the moisturizer and the oil augmenter from spreading and promoting the growth of microbes.

19. The preparation of claim 17, wherein the oil augmenter is jojoba oil.

20. The preparation of claim 17, wherein the moisturizer is squalane.
